(19) United States
(12) Patent Application Publication
(10) Pub. No.: US 2018/0330805 A1
(43) Pub. Date: Nov. 15, 2018

(54) COHORT EXPLORER FOR VISUALIZING COMPREHENSIVE SAMPLE RELATIONSHIPS THROUGH MULTI-MODAL FEATURE VARIATIONS

(71) Applicant: KONINKLIJKE PHILIPS N.V., Eindhoven (NL)

(72) Inventors: Yee Him Cheung, Boston, MA (US); Yong Mao, Hawthorne, NY (US); Nevenka Dimitrova, Pelham Manor, NY (US); Nilanjana Banerjee, Armonk, NY (US); Johanna Maria de Bont, Eindhoven (NL); Jozef Hieronymus Maria Raijmakers, Eindhoven (NL); Kostyantyn Volyanskyy, Larchmont, NY (US)

(21) Appl. No.: 15/973,775

(22) Filed: May 8, 2018

Related U.S. Application Data
(60) Provisional application No. 62/504,112, filed on May 10, 2017.

(51) Int. Cl. G16H 10/60; G16H 15/00; G06T 11/20

(52) U.S. Cl. CPC G16H 10/60; G16H 15/00; G06F 3/04817; G06T 2200/24; G06T 2210/41; G06T 11/206

(57) ABSTRACT

A data-driven integrative visualization system and a method for visualization and exploration of the multi-modal features of a cohort of samples, is disclosed Specifically, a method for providing an interactive computation and visualization front-end of a genomics platform for presenting the complex multiparametric and high dimensional, multi-omic data of a patient with respect to a cohort of samples, that assists the user in understanding the similarities and differences across individual or groups of samples, identify correlation among different features and improve treatment planning and long term patient care, is described. The method may include obtaining and inputting multi-omic data of a patient and/or cohorts, identifying multi-modal feature variations and their relationships, and displaying this information in an interactive circular format on a GUI, from which the user can access further information. The system may provide an improved process of integrative analysis on a patient's multi-omic data for effective treatment planning.

### CROSS REFERENCE TO RELATED APPLICATIONS

This application claims priority to U.S. Provisional Patent Application No. 62/504,112, filed on May 10, 2017, the entire disclosure of which is hereby incorporated herein for all purposes.

### TECHNOLOGICAL FIELD

Various exemplary embodiments disclosed herein relate generally to a cohort explorer for visualizing comprehensive sample relationships through multi-modal feature variations.

### BACKGROUND

Various visualization methods exist that present patient clinical data and lifestyle (quality of life) data which include results from a single modality measurement, for example a waterfall plot showing gene expression levels on a single gene, or tables in patient charts using EMR data. However, it is very difficult to show where a single patient is situated with respect to many parameters from clinical and genomic data and other data representative of the quality of life, where the patient data is sourced from a multitude of information sources in the clinical (e.g., Electronic Medical Record—EMR, and genomics information system) and personal life (e.g., quality of life inferred from quantified self-devices and social media) domains

### SUMMARY OF THE INVENTION

Embodiments described herein provide an improved presentation for exploration and comparison of multi-modal features of cohort samples with patient-oriented omic data (genomic, transcriptomic, proteomic, epigenomic, etc.), and patient-oriented information on social, economic, environmental, scientific, engineering, or any other types of data. In particular, Embodiments described herein include a system and method that provide an interactive visualization tool for summarizing and presenting patient and cohort data. Further, embodiments described herein provide interactive access to underlying intergenic genomic information, methylation and gene/exon expression data, on a genic scale, and nucleotide sequence, amino acid sequence and methylation data, on a molecular scale.

Thus, embodiments described herein provide a visualization tool, method and system for presenting and visualizing relevant patient-specific genomic and cohort information, such tool comprising, at a top level:

a sample inter-relationship plot in the middle, with each sample represented by a dot, and their distances and classifications depicted; and

multiple plots of selected features concatenated next to each other on the perimeter (e.g. a circular rim), each showing the variation profile of a feature for the cohort of samples; and

a sample information panel that contains the general information of the primary sample and the cohort.

Another embodiment includes a system and method comprising:

a computing device with a graphical user interface,

determining a dataset of files containing patient information, and storing said patient dataset on a server configured to store said dataset;

determining selection criteria based on the patient dataset;

inputting patient-specific data, by a user interface, onto a processor configured to receive said patient-specific data,

applying the selection criteria based on the patient dataset to determine a dataset of files containing cohort information, and storing said cohort dataset on a server configured to store said dataset;

comparing said patient dataset with said cohort dataset based on said selection criteria;

generating and displaying a visualization plot containing said patient dataset information and said cohort dataset information on a graphical user interface;

predicting subtype probabilities; and

administering a treatment protocol based on said subtype probabilities and the state of the patient as revealed by a combination of feature values.

Various embodiments relate to a computer-implemented method for visualization and exploration of multi-modal features of a cohort of patient samples, the method including:

generating a patient inter-relationship plot based upon at least two patient inter-relationship values; displaying the patient inter-relationship plot on a graphical user interface; wherein the patient inter-relationship plot comprises a plot of patient inter-relationship values for each patient, with each of the patient inter-relationship values represented by a patient icon; a perimeter of said the patient inter-relationship plot comprising multiple feature plots of selected features, each of the feature plots on the perimeter showing the variation profile of a feature for each of the patient samples; and a sample information panel adjacent said patient inter-relationship plot displaying patient sample information.

Various embodiments are described, wherein the patient inter-relationship plot includes: a selected patient icon for a selected patient; a patient feature value indicator on each of the feature plots for the selected patient; and multiple display lines connecting the selected patient icon to each of the patient feature value indicators.

Various embodiments are described, further including: receiving an input from the user selecting a specific feature value indicator; and displaying the value associated with the specific feature value indicator.

Various embodiments are described, wherein the patient inter-relationship plot further includes: a sub-perimeter comprising multiple feature plots of selected features, each of the feature plots on the sub-perimeter showing the variation profile of a feature for each of the patient samples; a patient feature value indicator on each of the feature plots on the sub-parimeter for the selected patient, wherein the multiple display lines further connect the selected patient icon to each of the patient feature value indicators at the sub perimeter, and wherein the feature plots on the perimeter and the sub-perimeter are taken at different times.

Various embodiments are described, wherein patient sample information in the sample information panel corresponding to the selected patient is highlighted.

Various embodiments are described, wherein the patient icons are grouped according to a subtype, and each group is indicated and labeled.

Various embodiments are described, further including receiving input from a user indicating cohort criteria for selecting patient samples to form the cohort of patient samples.

Various embodiments are described, further including receiving input from a user indicating which feature plots to display.

Various embodiments are described, further including receiving input from a user indicating the locations of the feature plots to display.

Various embodiments are described, further including: receiving input from a user a selecting a specific feature plot; and displaying an expanded instance of the specifed feature plot.

Various embodiments are described, wherein the feature plots grouped in segments along the perimeter according to feature groupings.

Various embodiments are described, further includes receiving input from a user selecting at least two different patient icons wherein the patient inter-relationship plot includes: a selected patient icon for each of the selected patients; a patient feature value indicator on each of the feature plots for each of the selected patients; and multiple display lines connecting each of the selected patient icons to each of the associated patient feature value indicators.

Various embodiments are described, wherein the feature value indicators and multiple display lines associated with each selected patient icon have different visual schema.

Various embodiments are described, wherein at least two patient inter-relationship values indicate a similarity distance between patients.

Various embodiments are described, wherein at least two patient inter-relationship values indicate a clustering of patients by subtype.

Various embodiments are described, further including: additional patient inter-relationship plots wherein the patient inter-relationship plots are displayed in 3-dimensions where each patient inter-relationship plot is a layer in the display, wherein the additionally patient inter-relationship plots are for different patients and/or cohorts of patient samples.

Various embodiments are described, further including: receiving a user selection selecting one of the patient inter-relationship plots; and displaying only the selected patient inter-relationship plot.

Various embodiments are described, further including receiving input from a user indicating a switch to a tile view, wherein each of the feature plots are additionaly presented in a separate tile.

Various embodiments are described, further including receiving input from a user selecting a plurality patient icon; performing a statistical anlysis on the patient sample data for the selected patient icons; and displaying a single combined patient icon on the patient inter-relationship plot using the results of the statistical analysis in place of the plurality of selected patient icons.

Various embodiments are described, further including receiving input from a user indicating that the user is hovering over a specific patient icon; and while the user indication is received displaying a patient feature value indicator on each of the feature plots for the specific patient icon and multiple display lines connecting the selected patient icon to each of the patient feature value indicators.

### BRIEF DESCRIPTION OF THE DRAWINGS

The methods according to the invention will now be described in more detail with regard to the accompanying figures. The figures showing ways of implementing the present invention and are not to be construed as being limiting to other possible embodiments falling within the scope of the attached claims.

FIG. 1 shows a flowchart of the key components and processing steps of Cohort Explorer.

FIG. 2 shows an embodiment of the Cohort Explorer of the within invention.

FIG. 3 shows the Cohort Explorer with feature plots arranged in tiled rectangular panels.

FIG. 4 shows the feature plots of a patient arranged in multiple concentric rings. In this example, each ring summarizes the status of a patient at each time point.

FIG. 5 is a detailed view of the gene signature results of the patient of interest, which includes subtype probabilities (top left), survival curves of each subtype (top right) and a heatmap (bottom) that shows the gene signature expressions of the samples.

FIG. 6 shows the Cohort Explorer in multiple layers for comparison between different samples and cohorts, with each feature vertically aligned across the layers.

FIG. 7 illustrates extended categories of clinically relevant multi-modal data that can be presented in Cohort Explorer.

FIG. 8 shows the integration of quality-of-life data with other categories of multi-modal data for presentation in Cohort Explorer.

### DETAILED DESCRIPTION OF THE INVENTION

The embodiments described herein relate to a data-driven integrative visualization system and a method for visualization and exploration of the multi-modal features of a cohort of samples. Specifically, a method for providing an interactive computation and visualization front-end of a genomics platform for presenting the complex multiparametric and high dimensional, multi-omic data of a patient with respect to a cohort of samples, that assists the user in understanding the similarities and differences across individual or groups of samples, identify any correlation among different features and improve treatment planning and long-term patient care, is described. The method includes obtaining and inputting multi-omic data of a patient and/or cohorts, identifying multi-modal feature variations and their relationships, and displaying this information in an interactive format on a GUI, from which the user can access and view further information. The medical practitioner is able to access underlying supporting biologic and scientific evidence from relevant knowledge bases through a set of graphical interactions. The system provides an improved process of integrative analysis on a patient's multi-omic data in conjunction with cohort samples for effective treatment planning.

Various visualization methods exist that present patient clinical data and lifestyle (quality of life) data which include results from a single modality measurement, for example a waterfall plot showing gene expression levels on a single gene, or tables in patient charts using EMR data. However, it is very difficult to show where a single patient is situated with respect to many parameters from clinical and genomic data and other data representative of the quality of life, where the patient data is sourced from a multitude of information sources in the clinical (e.g., Electronic Medical Record - EMR, and genomics information system) and personal life (e.g., quality of life inferred from quantified self-devices and social media) domains. Exploring and visualizing the patient and genomic data may be performed using various mathematical approaches. The problem is that genomic data is inherently high-dimensional data. For the purpose of visualization, methods exist also for reducing dimensionality of the original multiparametric space so that the clustering/relatedness of the samples could be seen in a new transformed space. Methods that are able to reduce the dimensionality are principal component analysis, multidimensional scaling, etc. In addition, many of the clinical parameters are on different scales, and it is not easy to normalize all the data in order to show where an individual patient is positioned with respect to other patients.

Thus, in both clinical and research settings, it is often useful to visualize and compare samples based on a set of characterizing feature values, which could be of diverse nature. The complexity arises because the genomic data comes from multiple modalities (e.g., mutations, gene expression, epigenomic differences), and their similarities/differences across modalities and identify any correlations among the different features need to be understood. In the embodiments described herein, a novel tool is provided which is designated the “Cohort Explorer,” for the effective visualization and exploration of the multi-modal features of a cohort of samples that can help clinicians/scientists disentangle sample relationships and gain insight into the underlying factors or mechanism that drive the clinical or phenotypic differences across individual or groups of samples. Although the functionalities of Cohort Explorer are illustrated in the context of clinical, biological and genomic data, the embodiments described herein are broadly applicable to the comparison of samples based on social, economic, environmental, scientific, engineering, or other types of data.

In comparison, a “Heatmap” is a popular tool for visualizing multiple quantitative features, usually gene expressions, across samples. With proper clustering, the underlying structure/pattern of the features and their associations with specific groups/subtypes of samples can be systematically revealed. However, the Heatmap is primarily designed for the presentation of homogeneous features, and the use of a color scale is less precise for visual comparison. Moreover, the two-dimensional matrix layout is inflexible in that it requires all features to be shown in the same sample order, making it difficult to inspect the different rankings of a sample with respect to separate features. In contrast, the embodiments described herein allows for visualization of multiparametric data across a wide variety of clinical domains and across large cohorts of patient data.

The embodiments of the Cohort Explorer described herein provide various technological improvements and advantages. The Cohort Explorer visualizes a complete patient record (data structure) with very complex patient data of various categories pulled from various information systems in the clinic and the personal sphere of life of the patient. On the clinical side, the visualization system may pull information from Electronic Medical Record, Laboratory Information System, Pharmacy Prescription System, outpatient systems, and cancer registry databases. On the personal side, the information may be obtained from “quantified self” devices, health watch (like Apple's Apple Watch) and various activity monitoring devices such as Fit-Bit. In addition, the system (with proper permissions and business level agreements) can pull information from various Applications (“Apps”), on the patient's phone, that represent patient's activity, mood or vital status. For example, number of tweets, number of likes on FaceBook, use of emojis etc.

The Cohort Explorer also visualizes sample distance or classification, and relates each sample to a specific set of feature values. Further, the Cohort Explorer supports the presentation of multi-modal or heterogeneous features. The Cohort Explorer also provides the flexibility for adopting different types of plots, styles, formats or sample ordering for different features as required by the user. Finally, the Cohort Explorer supports a rich set of interactions to assist users in exploring sample relationships and detailed data.

The Cohort Explorer may be implemented as a standalone application, a web-based application, mobile device application, or a GUI component that takes processed omic and other data as inputs. Besides visualizing and presenting the data, the tool also accepts user inputs and interactions, and queries different knowledge bases to incorporate further information when desired.

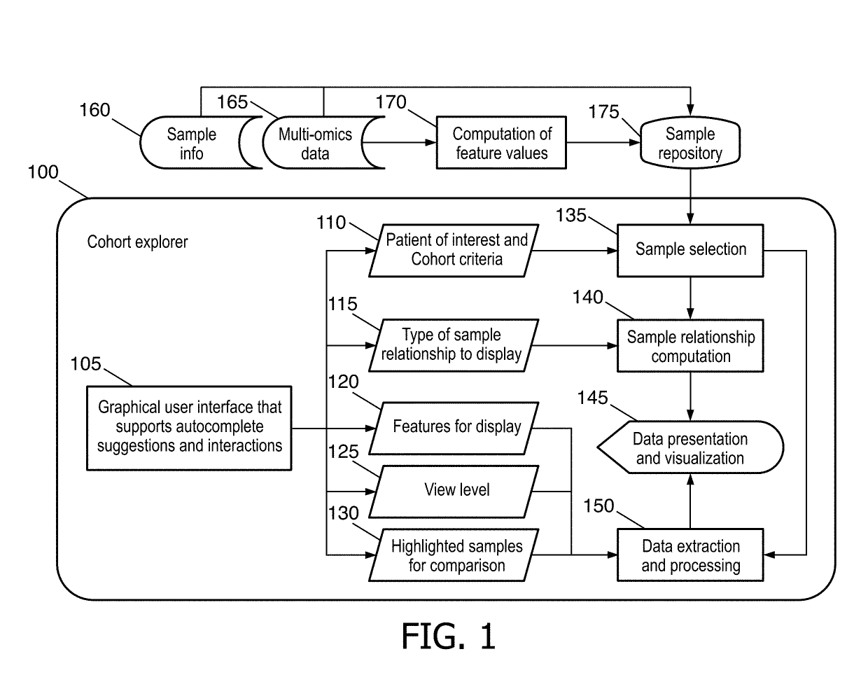

FIG. 1 shows a flowchart of the key components and processing steps of Cohort Explorer. A sample repository 175 stores medical data for patients for use by the Cohort Explorer 100. Sample information 160 may be directly stored in the sample repository 175. Further, sample information may be used to determine multi-omics data 165. The multi-omics data 165 may be used to compute feature values 170. The computed feature values 170 may also be stored in the sample repository.

The Cohort Explorer includes a graphical user interface 105. The graphical user interface 105 may support autocomplete suggestions and interactions. The graphical user interface 105 allows a user to provide inputs to determine what specific information will be displayed by the Cohort Explorer 100. The graphical user interface 105 provides the user great flexibility in configuring the information displayed by the Cohort Explorer 100. The graphical user interface 105 may receiving information indicating the patient of interest and cohort criteria 110. The patient of interest and cohort criteria 110 are used by the sample selection module 135 to produce a sample selection from the sample repository 175. This sample selection may then be input into the sample relationship computation module 140 and the data extraction and processing module 150. The sample relationship computation module 140 may also receive information regarding the type of sample relationship to display 115. The sample relationship computation module 140 then processes the various received data to produce an output that may be used to present various specified data in the requested formats. This output is then received by the data presentation and visualization module 145 that produces the final output data and signals to be presented on a display for the user. Additionally, the data presentation and visualization module 145 also received data from the data extraction and processing module 150 for display. The data extraction and processing module 150 receives data from the sample selection module 135 as well as information relating to features to display 120, view level 125, and highlighted samples for comparison 130. The features for display 120 indicate which data features are to be displayed and in what type of format. The view level 125 indicates the view levels for the data as will be explained below. The highlighted samples for comparison 130 indicate which specific samples, e.g., specific patients, have been highlighted by a user that will then display more specific information for those specific samples. As a user interacts with the display presented by the Cohort Explorer 100, various aspects of this flowchart will come into action.

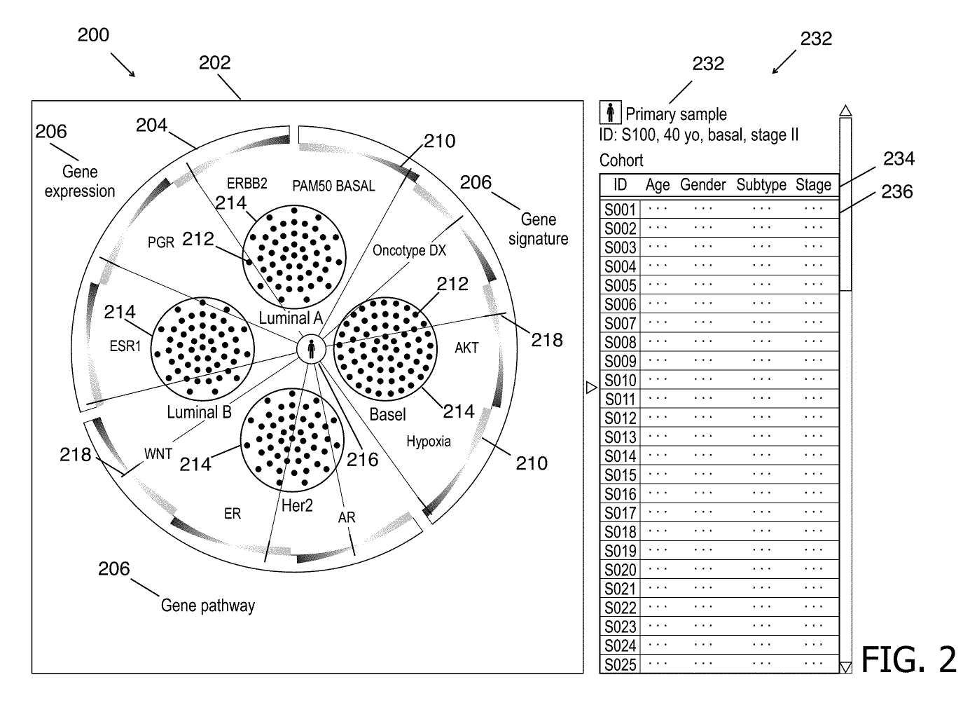

FIG. 2 shows an embodiment of the Cohort Explorer. Specifically, in FIG. 2 for example, the Cohort Explorer presents breast cancer data for a number of patients. The Cohort Explorer 200 includes various elements including the patient inter-relationship plot 202 and sample data panel 230. The patient-inter-relationship plot 202 is illustrated as a two-dimensional plot that places patient icons 212 for each patient in a cohort on the plot based upon the values of two features found in the patient data. In other embodiments, higher dimensional data may be displayed on the patient inter-relationship plot 202. This data may be derived in various ways. For example, they may simply be two feature values recorded for each patient. The two values may be the result of principal component analysis (PCA), machine learning processing, auto encoding, neural network processing, etc. In some embodiments, the patient inter-relationship plot 202 may indicate the distance between patients or the sameness of patients based upon the two values. Then as shown in FIG. 2 various clusters of patients 214 are shown with the clusters indicating different types of breast cancer such as Luminal A, Luminal B, Base1, and Her2. The patient inter-relationship plot 202 is shown with a circular perimeter 204. Arranged along this perimeter are feature plots 210 of various features. These feature plots 210 in this specific example are shown as waterfall plots, but as described below, these plots may take other forms for presenting data for the patients. Each of these plots may include a plot label 208 indicating the feature illustrated. Additionally, the feature plots 210 may be grouped and labelled 206, such that the groups provide related information. For example, in FIG. 2, the groups Gene signature, Gene pathway, and Gene expression are shown. A primary sample of interest 216 may also be selected. Upon such selection, connection lines 220 may be displayed radiating from the primary sample of interest 216 out to various feature plots 210, where an indication 218 of the value of that specific features for the primary sample of interest 216 is illustrated. More detail regarding the Cohort Explorer 200 will now be provided.

The primary sample of interest 216 may be represented by an icon such as a human figure and characterized by a distinctive set of multi-modal feature values, with respect to the variation profiles of any cohort of samples for comparison. The features values associated with the primary sample 216 are marked using feature value indicators 218 on the respective feature plots 210 at the perimeter, with optional connection lines 220 showing the connections between them. Importantly, the patient cohort (as identified by the various patient icons 212) that is visualized in the center of the patient inter-relationship plot 202 is the same as the cohort in the feature plots 210 represented at the perimeter of the patient inter-relationship plot 202. Here, a feature could be quite complex. It could represent levels of gene expression. It could also represent recurrence scores such as the scores reported by OncotypeDx from Genomic Health. These values may range from 0 to 60. Another type of feature could be the values from the mutational tumor burden from each sample. Also, the features may comprise values from predictive scores for response to specific therapy: for example, probability of response to an anti-angiogenic drug called bevacizumab.

Specifically, FIG. 2 is an example of a Cohort Explorer 202 for breast cancer samples, which are represented by the patient icons 212 and grouped by their intrinsic subtypes 214. The groups 214 may be indicated by a perimeter drawn around the group. On the perimeter 204 of the patient inter-relationship plot 202 are multiple feature plots 210 that are, in this example, waterfall plots of selected features in the categories of gene expression, signature, and pathway. The feature values of the sample of interest 216, highlighted by the human figure, are marked by a feature value indicator 218 in the respective waterfall plots with connection lines 220 connecting each of the feature value indicators 218 to the sample. General information of the primary sample and cohort is shown in the sample panel data 230 on the left.

In this example, the relationships of the primary and cohort of samples are depicted in the patient inter-relationship plot 202, with each sample represented by a patient icon 212. Other data plotted by the patient inter-relationship plots 202 may include but are not limited to:

1. Distance-based—multidimensional scaling (MDS) plot, principal component analysis (PCA) plot, or any combination of quantitative sample attributes (e.g., demographic attributes like age, body mass index, physiological measurements like heart rate, blood pressure, metabolism measurements such as glucose/cholesterol level, genomics results like SNPs and functional readouts like transciptomics etc.);

2. Cluster-based—samples grouped by different subtypes generated by unsupervised learning methods (e.g., hierarchical clustering, k-means clustering) or classifications marked by color, symbol or boundary lines;

3. Hybrid—a mix of distance- and cluster-based approaches: where the clusters will be displayed as separate groups, but within each group the distance-based methods will determine how closely or how far samples are from each other in the distance space; and

4. Other—any other technique that takes multidimensional patient data and produces lower dimensional patient data that indicates the inter-relatedness among the patients.

FIG. 2 also illustrates feature plots 210 in a circular layout. The user selects a set of heterogeneous or multi-modal features, each of which is represented as a separate feature plot 210 that summarizes the features variation across all patient samples. The features may also be selected by default in the system. One useful type of plot for displaying quantitative feature values is a waterfall chart, which is basically a bar chart with samples ordered in increasing/decreasing feature values and optionally grouped by their subtypes, which can be denoted by the bar colors. For plots arranged in such a circular layout, one way to avoid any confusion on the direction of the y-axis for plots in the bottom half of the circle is to impose the convention that the positive direction is pointed radially outward from the center of the circle. Other types of charts, such as scatter, line, pie, box, or radial charts, can also be used to display the feature values as appropriate.

In addition, the Cohort Explorer 202 may take many shapes. Instead of a circular layout the separate plots of features may be represented in a linear, dual, triangle, quadrilateral, pentagonal, hexagonal, etc. shape of layout.

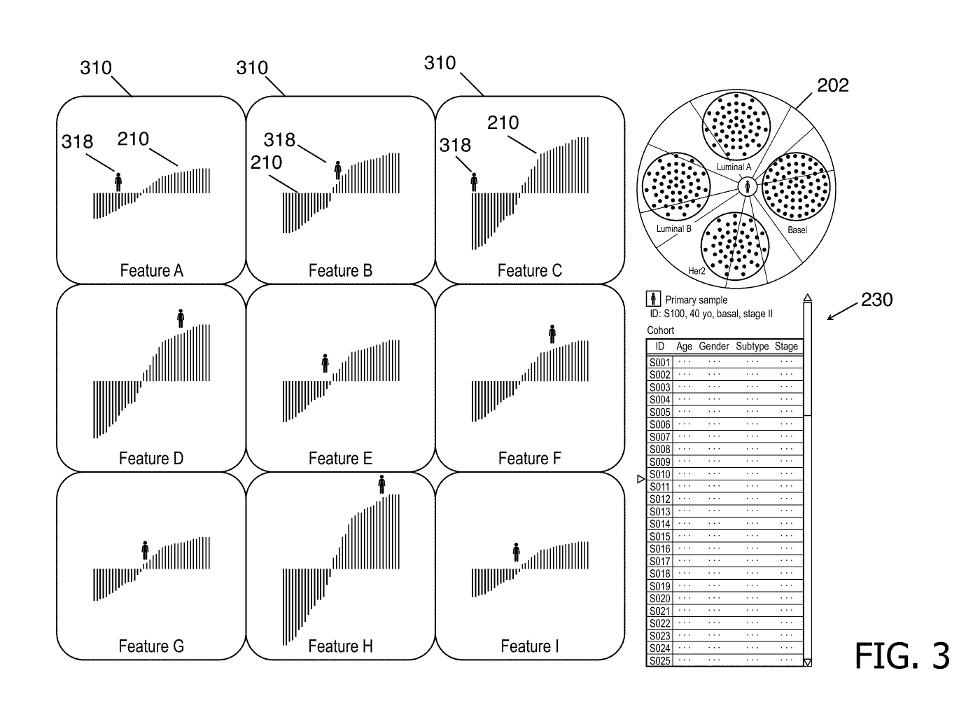

The user may select an alternate view for viewing the specific feature plots 210. For example, FIG. 3 shows the Cohort Explorer 202 with feature plots 210 arranged in tiled rectangular panels 310. Each feature plot includes a patient icon 318 positioned to indicate the specific patient's value on the feature plots 210. Such a view provides an alternate view of the feature data for a patient that allows the user to see different relationships in the data.

Users also may manage the order of the feature plots 210 and organize them into segments under different categories 210. In FIG. 2, for example, the features are grouped into three categories: gene expression, signature and pathway. For each category, the waterfall chart shows respectively the gene expression level, probability of the predicted subtype of a gene signature and the predicted activity of a signaling pathway. Users may choose to show/hide a group of feature plots by clicking on the category title, switching the layout from one shape to another by selecting the shape, and in the case of a circular layout rotate the wheel by swiping

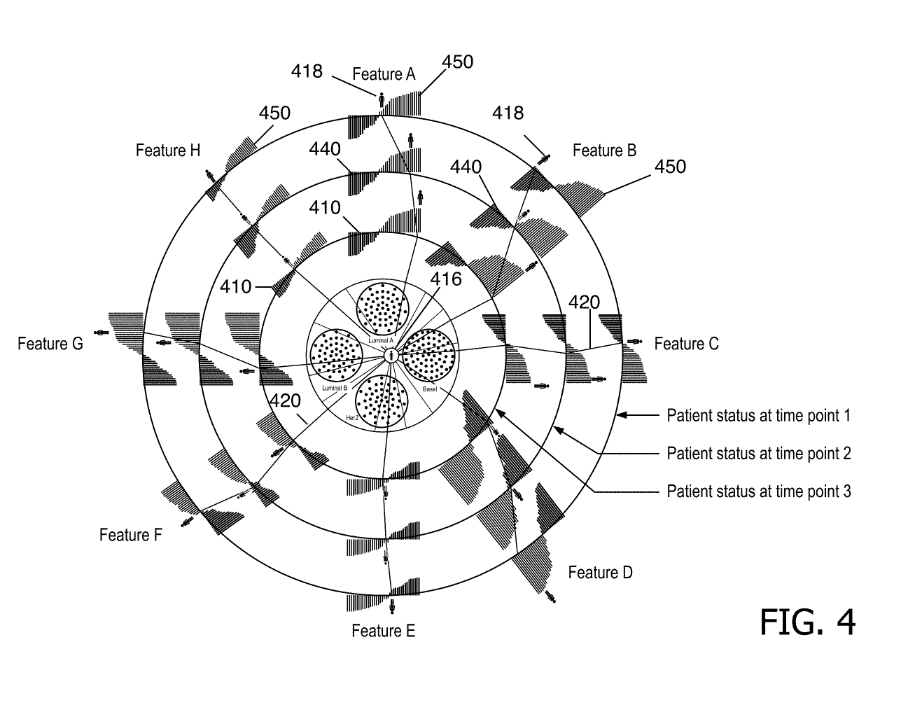

FIG. 4 illustrates another view for the Cohort Explorer. In this view the feature plots are organized into multiple concentric-rings show a time progression for the various feature plots. For example, the outer ring of feature plots 450 are the feature plots at time point 1. Likewise, the other two rings of feature plots 440 and 410, are the feature plots at time points 2 and 3 respectively. Like in FIG. 2, lines 420 extend from the sample of interest 416 and cross the feature plots in the different rings at the values for the sample of interest 416 at the different time points. Additionally, an icon 418 may be placed adjacent to the feature plots to indicate corresponding value for the sample of interest. Such a view in the Cohort Explorer allows a user to gain insights to the data based upon a time progression. The user has the flexibility to determine the number of time points to be displayed as well as the specific time points to use. For example, the Cohort Explorer may present a list of time points for which the feature data is available, and then the user selected the specific times to display. Further, the additional rings may display the feature variations of a particular subset of samples, e.g. different perimeters for Luminal A, Luminal B, HER2+ and Basal subtypes of patients, where each perimeter may be in a color that matches the color of the subtype. In another embodiment, the extra rings may be used to display additional feature plots, when a single ring cannot accommodate all of the feature plots to be displayed.

The sample data panel 230 shown in FIG. 2 provides additional sample information. General information, such as ID, age, subtype, status, etc. 234, of the primary sample 232 and each of the cohort of samples 236 may be displayed in the scrollable panel. Samples selected in the plot may be highlighted in the panel by a different visual schema such as text/background color of their corresponding records. Specifically, samples with their feature values highlighted in the plot may be marked accordingly with their designated symbols and colors alongside their records in the panel. The information panel may be expanded or hidden by clicking on an open/close button.

To investigate the similarities and differences between multiple samples, users select/unselect one or more samples by clicking on the individual samples or selecting a region in the sample inter-relationship plot. The selected samples are then highlighted, with their feature values and the connection lines 220 may be marked distinctively using different visual schema for each sample by using different colors or marker symbols. In this manner, if the features are close in the patient inter-relationship plot 202 and also close in the feature space—as shown in the feature plots 210 at the perimeter, then the user may conclude that these are similar patients. However, if the patients are close in the patient inter-relationship plot 202 and are only close on two out of the ten peripheral feature plots 210, then the user may conclude that these two patient samples have divergent features (i.e., different profiles) and the user (oncologist) cannot expect them to have similar outcomes. This in itself is quite informative.

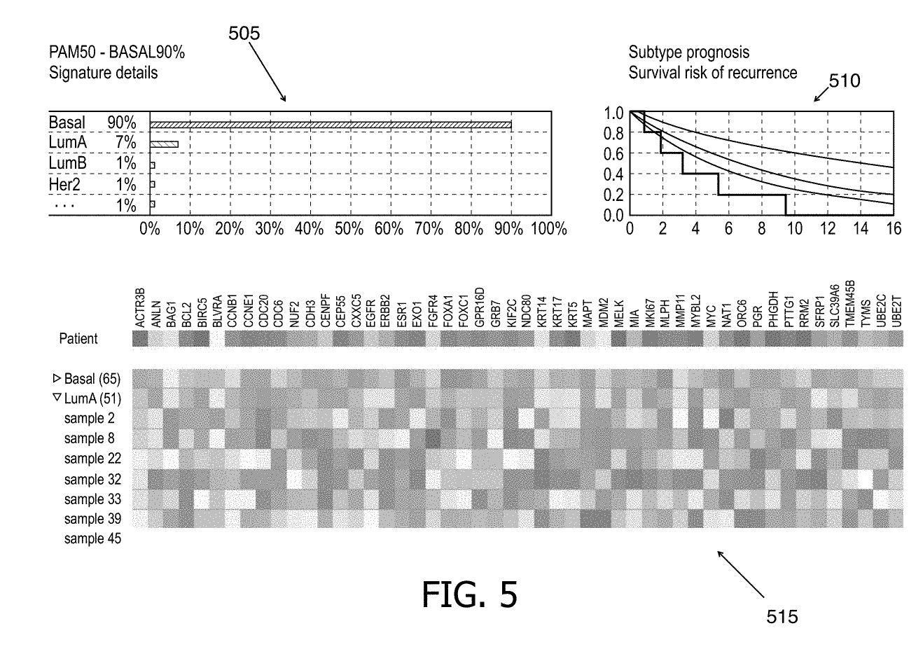

In exploring and comparing a particular feature, users may zoom into the specific feature (e.g., a signature plot), and explore its underlying details by clicking on its feature plot. From there, the user may directly navigate to the next/previous feature by flipping right/left. The content of the detailed view depends on the feature type. Within the Cohort Explorer system, there is a non-relational database that holds both the data structure for the related underlying data, as well as the functions that display clinically informative information on the data held in this structure. For example, FIG. 5 is a detailed view of the gene signature for the classification of breast cancer samples for a patient of interest, which includes a subtype probabilities plot 505, survival curves of each subtype 510, and a heatmap 515 that shows the gene signature expressions of the sample. In order for FIG. 5 to show the detailed view of the gene signature and the survival analysis, the following are needed: 1) the predicted subtype probabilities which are computed from gene expression data from the patient, using a very specific signature decision function (e.g., closeness to a cluster centroid), or in a second case, data extracted from a pdf report retrieved from the patient data structure and displayed in the subtype probabilities plot 505; 2) the survival curves of each subtype 510 computed from progression-free survival or overall survival data using Kaplan-Meier plotting, and the corresponding gene expression heatmap 515 from the genes in the set of signature genes. From the subtype probabilities plot 505, the clinician may understand the probability of a patient belonging to a certain cancer subtype. Once a subtype is identified on from the subtype probabilities plot 505, then this cancer subtype is associated with a survival profile. For example, if the probability of the patient belonging to the basal subtype profile is high, then, the survival curve 510 shows the worst prognosis for this subtype (the bottom of the three survival curves). If a clinician is interested in the gene expression profile for this particular subtype, the heatmap 515 will show each of the genes and its expression value for this particular patient, then for the specific subtype that the patient belongs to, and then expression values for all the samples in the cohort—for comparative purposes.

To improve a user's visualization experience and provide a 3D look and feel, an embodiment of the Cohort Explorer also includes a 3D sample inter-relationship plot, with the feature plots in upright positions and around a layout in the horizontal plane.

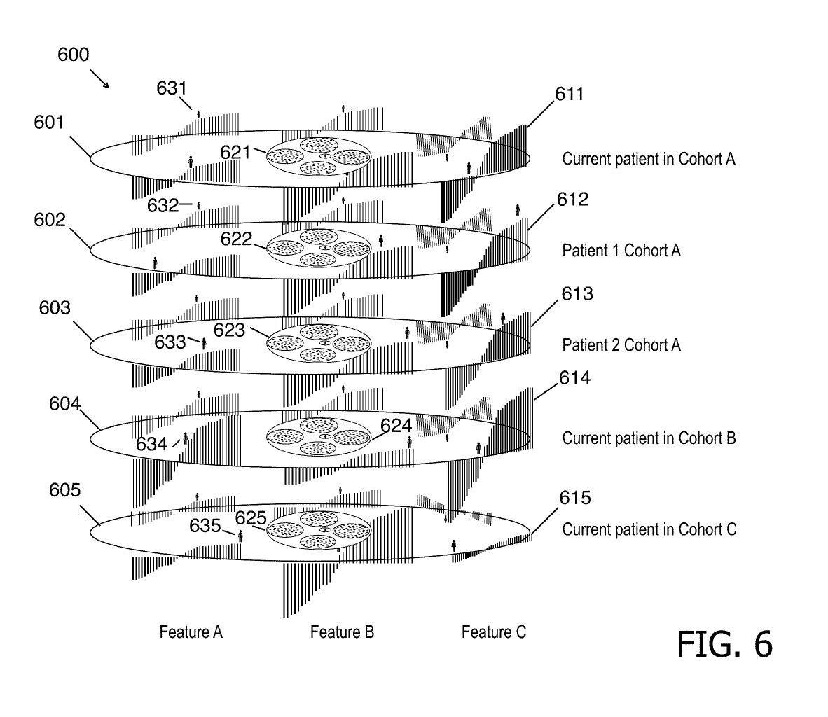

To facilitate the comparison between samples and cohorts, the Cohort Expander can be further extended to support multiple layers in a vertical/horizontal stack, with each layer representing a different cohort of samples and the same feature aligned and locked across layers. FIG. 6 illustrates a 3D layered Cohort Explorer 600. The 3D layered Cohort Explorer 600 includes layers 601, 602, 603, 604, and 605. The layers present various patients in various cohorts. For example, layer 601 shows the current patient in cohort A, layer 602 shows patient 1 in cohort A, layer 603 shows patient 2 in cohort A, layer 604 shows the current patient in cohort B, and layer 605 shows the current patient in cohort C. Each of the layers include respective patient inter-relationship plots 621, 622, 623, 624, 625 with feature plots 611, 612, 613, 614,615 around the perimeter. As in the other embodiments, patient icons 631, 632, 633, 634, 635 are placed by the feature plots to show the specific patient values on the feature plots. A user may select as specific layer which then replaces the 3D layer view with just a view of the specific layer. Also, the user may select different feature plots, which are then displayed in detail like in the embodiments described above. Also, the user can add or delete layers and change the cohorts and/or patients selected to be displayed in the various layers.

Various user inputs and interactions of the Cohort Explorer will now be described. The Cohort Explorer also provides a set of interaction capabilities to facilitate the user's exploration of sample relationships and provide quick access to detailed data and additional resources. User interactions include but are not limited to the following:

### Data Import and Cohort/Sample Selection

1. Import the data files of the cohorts of samples with their clinical information

2. Import the features with their type definitions (e.g., gene expression, signature or pathway), their values for each sample, and any other supporting data to be displayed in the detailed view

3. Select a cohort of samples based on specific criteria, such as name of study, tissue types, sample demographics, clinical phenotypes, treatment history, etc.

4. Designate one primary sample of interest whose data serves as the reference for comparison, with feature values and associated connections highlighted by default Sample Inter-relationship Plot Related

5. Select the type of sample relationship, such as MDS, PCA, subtype clustering, etc., to be displayed

6. Select/Unselect one or multiple samples by clicking on them individually or choosing a region. Then a list of applicable actions, such as highlight, delete, collapse, new subtype, reset, etc., will appear for selection by the user. The records of any selected samples will be highlighted in the sample information panel. Unselect all by clicking on any space.

7. For the first several samples selected, their symbols, feature values and connection lines will be marked and highlighted. Different visual schema such as colors/symbols may be applied to distinguish the samples and their data from each other. These samples will also be marked by their specific colors/symbols in the sample information panel.

8. By hovering over a sample point, the sample, its associated feature values and connections, and a box with brief information about the sample will be shown and highlighted.

9. Detailed information of a particular sample may be shown by double-clicking on its marker in the sample inter-relationship plot.

10. Multiple selected samples may be collapsed—replacing their individual feature values by the average, combining their markers into one symbol, and indicating the grouping in the sample information panel.

11. A new subtype may be defined and assigned to a set of selected samples, which are clustered and marked accordingly in the sample inter-relationship plot with their records updated in the sample information panel.

12. Move, rotate, or zoom in/out the sample inter-relationship plot

13. Move individual or a group of selected samples by dragging

14. Reset the plot/samples to the original settings based on the input data Sample Information Panel Related

15. Any interactions on the sample points in the plot described above can be equivalently applied to the sample entries in the information panel whenever appropriate.

16. Expand/hide the panel by clicking an open/close button

### Feature Plots Related

17. Select/add the features to be shown—one-by-one manually or using predefined sets of features for specific types of disease

18. For each feature, users can change the plot type, detailed view format (pre-designed or customized), section, rim level (concentric rims with higher levels for outer rims), etc. Otherwise, the default presentation settings are applied.

19. Reorder the feature plots on the circular rim or group them into different categories

20. Merge/split the feature plots into the same/different rims by selecting and dragging one or multiple plots

21. Show the detailed view of a feature by clicking on a plot

### Other

22. Add one or more layers of Cohort Explorer, and import a different set of data for each layer.

23. Merge/Split a selected cohort into the same/different layers of Cohort Explorers by dragging

24. Move, rotate, zoom in/out or change the visual perspective of an individual or a stack of Cohort Explorers

A few use cases of the Cohort Explorer for different diseases will now be described. In a first use case as illustrated in FIG. 2, an oncologist wants to compare a 40 year old, stage II, pathological HER2+ breast cancer patient with a cohort of breast cancer patients. The oncologist uses Cohort Explorer to explore and investigate where this patient stands among different subtypes of breast cancer patients in order to assist treatment planning.

The oncologist first imports into the Cohort Explorer data files that include samples with their IDs, demographic and clinical information, gene expression levels, predicted subtype probabilities of multiple gene signatures, predicted signaling pathway activities, etc. From the list of imported samples, the oncologist applies selection criteria so that only stage II patients of age between 40 and 50 are included for display. The oncologist designates their patient as the primary/reference sample and selects the set of features predefined for breast cancer: gene expression levels of ESR1/PGR/ERBB2, predicted activities of signaling pathways Wnt/ER/AR and predicted subtype probabilities of several gene signatures.

By default, PCA is performed on the gene expression data, and the relationships of the samples are depicted in a two-dimensional principal component plot. The oncologist further requires that the subtypes of the samples be indicated by different symbols and colors, and the samples are in general clustered by subtypes despite some overlaps and outliers. Their patient is found to lie in the border between the HER2+ and basal subtypes.

By looking at the waterfall plot of the ERBB2 gene expression, the oncologist finds that their patient is only marginally overexpressed for ERBB2 compared with other HER2+ patients, implying that the conventional treatment for HER2+ breast cancer may not be as effective for this patient. Moreover, gene signature prediction shows that the patient has a 60% chance of actually having the basal type of breast cancer. The oncologist further compares the gene expressions of the patient with the basal group and finds that the expression profile of their patient is comparable to that group.

Based on the waterfall plots of the predicted pathway activities, the oncologist finds that actually the patient has a Wnt pathway activity that is higher than 90% of all the breast cancer patients, hinting on the potential benefits of administering Wnt pathway inhibitors in the treatment of the patient.

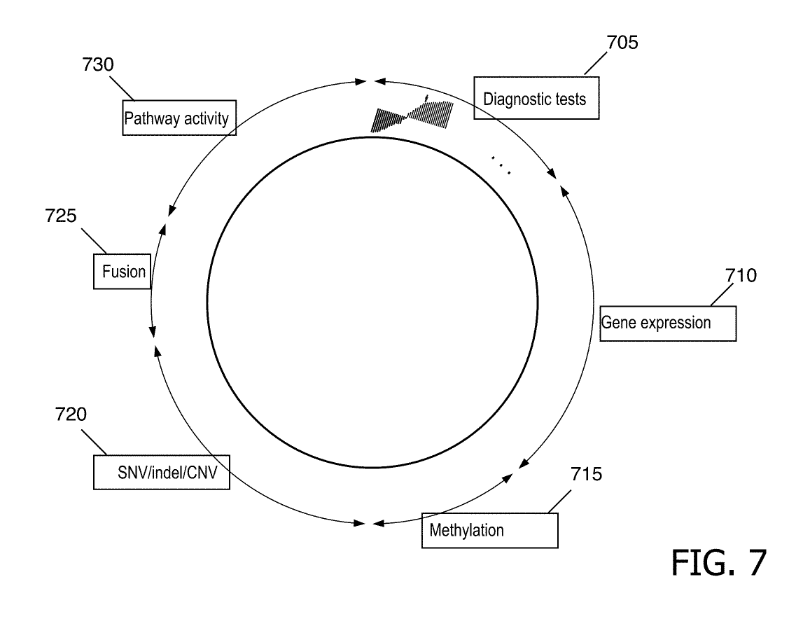

Although the application of Cohort Explorer for the presentation of breast cancer data is illustrated as an example above, the tool can easily be adapted for use on other cancer types or even non-clinical data with sample relationship or structure that needs to be explored. FIG. 7 illustrates the data structure with the possible types of data that could be summarized for a prostate cancer patient in the Cohort Explorer.

### Diagnostic Test Results 705

1. Gleason's Score—prognosis based on microscopic appearance

2. InformMDx—aggressive/non-aggressive

3. Oncotype DX—risk assessment score

4. NADiA ProsVue—risk for recurrence

5. Prolaris (Myriad)—risk of disease progression

### Related Multi-Omic Data

6. Gene Expression 710:
(a) Oncogene—AR
(b) Tumor Supressor—p53, PTEN, PCA3, ERG
(c) Emerging biomarkers—RAF, BRAF, SPOP, EZH2, Spink1

7. Methylation 715: PTEN

8. SNV/indel/CNV 720: AR, p53, CDKN1B, NKX3.1, PTEN

9. Fusion 725: TMPRSS2-ERG

### Signaling Pathway Activities 730

10. AR pathway activity, ER pathway activity, Wnt pathway activity, Hedgehog pathway activity, PI3K/FOXO, NFkB, TGFb, Notch, etc.

Related Personal and “Quality of Life” data

11. number of tweets (could be latest number or average per day)

12. number of likes on social media

13. emotional wellbeing expressed as average emoji type of icons entered into a social network

14. Game activity from an App from patient's phone

15. Emotional status based on the sentiment analysis using Natural Language Processing (NLP) for social tweets

This last group of data could reflect the overall status and impact of particular drug on the quality of life of the patient while being on a certain therapeutic regimen.

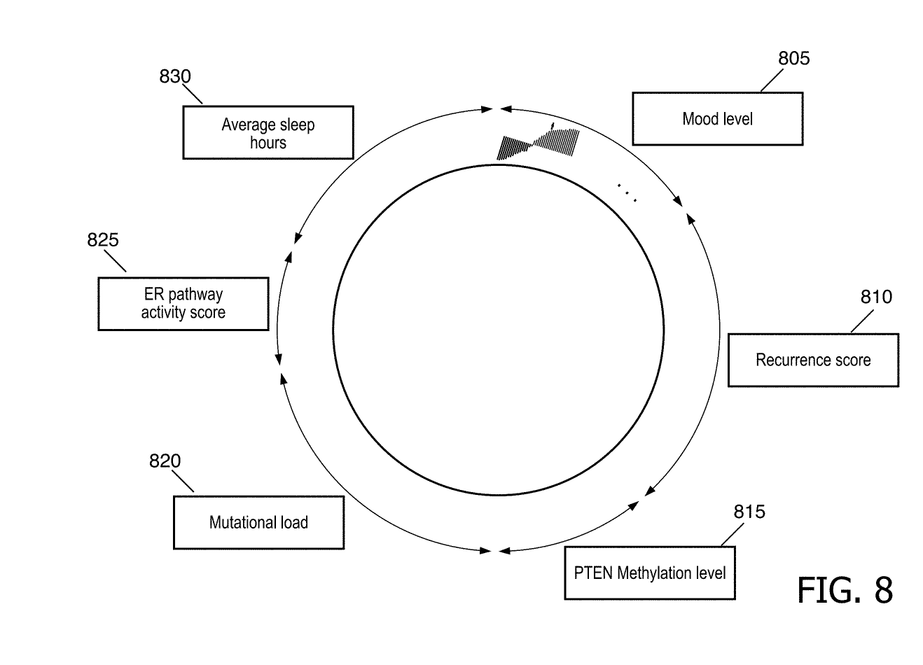

FIG. 8 illustrates an example how quality of life data may be visualized. Examples of quality of data may include average sleep hours 830 and mood level 805 (on a scale of 1 to 10). This quality of file date may be displayed along with other data such as a recurrence score 810,

PTEN Methylation level 815, a mutational load 820, and ER pathway activity score 825. There are studies that show association of these quality of life indicators with the treatment outcome.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed:

1. A computer-implemented method for visualization and exploration of multi-modal features of a cohort of patient samples, the method comprising:
generating a patient inter-relationship plot based upon at least two patient inter-relationship values;
displaying the patient inter-relationship plot on a graphical user interface;
wherein the patient inter-relationship plot comprises a plot of patient inter-relationship values for each patient, with each of the patient inter-relationship values represented by a patient icon;
a perimeter of said the patient inter-relationship plot comprising multiple feature plots of selected features, each of the feature plots on the perimeter showing the variation profile of a feature for each of the patient samples; and
a sample information panel adjacent said patient inter-relationship plot displaying patient sample information.

2. A computer-implemented method of claim 1, wherein the patient inter-relationship plot comprises:
a selected patient icon for a primary selected patient;
a patient feature value indicator on each of the feature plots for the selected patient; and
multiple display lines connecting the selected patient icon to each of the patient feature value indicators.

3. A computer-implemented method of claim 2, further comprising:
receiving an input from the user selecting a specific feature value indicator; and
displaying the value associated with the specific feature value indicator.

4. A computer-implemented method of claim 2, wherein the patient inter-relationship plot further comprises:
a sub-perimeter comprising multiple feature plots of selected features corresponding to the features plots on the perimeter, each of the feature plots on the sub-perimeter showing the variation profile of a feature for each of the patient samples at a different point in time from the feature plots on the perimeter;
a patient feature value indicator on each of the feature plots on the sub-parimeter for the selected patient,
wherein the multiple display lines further connect the selected patient icon to each of the patient feature value indicators at the sub perimeter.

5. A computer-implemented method of claim 2, wherein the patient inter-relationship plot further comprises:
a sub-perimeter, each comprising multiple additional feature plots of selected features, each of the feature plots on the sub-perimeter showing the variation profile of a feature for each of the patient samples;
a patient feature value indicator on each of the feature plots on the sub-parimeter(s) for the selected patient,
wherein the multiple display lines further connect the selected patient icon to each of the patient feature value indicators at the sub perimeter.

6. A computer-implemented method of claim 2, wherein patient sample information in the sample information panel corresponding to the selected patient is highlighted.

7. A computer-implemented method of claim 1, wherein the patient icons are grouped according to a subtype, and each group is indicated and labeled.

8. A computer-implemented method of claim 1, further comprising receiving input from a user indicating cohort criteria for selecting patient samples to form the cohort of patient samples.

9. A computer-implemented method of claim 1, further comprising receiving input from a user indicating which feature plots to display.

10. A computer-implemented method of claim 1, further comprising receiving input from a user indicating the locations of the feature plots to display.

11. A computer-implemented method of claim 1, further comprising:
receiving input from a user a selecting a specific feature plot; and
displaying an expanded instance of the specifed feature plot with further information.

12. A computer-implemented method of claim 1, wherein the feature plots grouped in segments along the perimeter according to feature groupings.

13. A computer-implemented method of claim 1, further comprises receiving input from a user selecting at least two different patient icons wherein the patient inter-relationship plot comprises:
a selected patient icon for each of the selected patients;
a patient feature value indicator on each of the feature plots for each of the selected patients; and
multiple display lines connecting each of the selected patient icons to each of the associated patient feature value indicators.

14. A computer-implemented method of claim 13, wherein the feature value indicators and multiple display lines associated with each selected patient icon have different visual schema.

15. A computer-implemented method of claim 1, wherein at least two patient inter-relationship values indicate a similarity distance between patients.

16. A computer-implemented method of claim 1, wherein at least two patient inter-relationship values indicate a clustering of patients by subtype.

17. A computer-implemented method of claim 1, further comprising:
additional patient inter-relationship plots wherein the patient inter-relationship plots are displayed in 3-dimensions where each patient inter-relationship plot is a layer in the display, wherein the additionally patient inter-relationship plots are for different patients and/or cohorts of patient samples.

18. A computer-implemented method of claim 6, further comprising:
receiving a user selection selecting one of the patient inter-relationship plots; and
displaying only the selected patient inter-relationship plot.

19. A computer-implemented method of claim 1, further comprising receiving input from a user indicating a switch to a tile view, wherein each of the feature plots are additionaly presented in a separate tile.

20. A computer-implemented method of claim 1, further comprising:
receiving input from a user selecting a plurality patient icons;
performing a statistical anlysis on the patient sample data for the selected patient icons; and
displaying a single combined patient icon on the patient inter-relationship plot using the results of the statistical analysis in place of the plurality of selected patient icons.

21. A computer-implemented method of claim 1, further comprising:
receiving input from a user indicating that the user is hovering over a specific patient icon; and
while the user indication is received displaying a patient feature value indicator on each of the feature plots for the specific patient icon and multiple display lines connecting the selected patient icon to each of the patient feature value indicators.
